(19) United States
(12) Patent Application Publication
(10) Pub. No.: US 2024/0060035 A1
(43) Pub. Date: Feb. 22, 2024

(54) COLLECTING SPORES WITH HYDROPHOBIC LIQUID AND CORRESPONDING USE OF COLLECTED SPORES

(71) Applicant: The University of Akron, Akron, OH (US)

(72) Inventor: Lu-Kwang Ju, Akron, OH (US)

(21) Appl. No.: 18/235,573

(22) Filed: Aug. 18, 2023

Related U.S. Application Data
(60) Provisional application No. 63/399,445, filed on Aug. 19, 2022.

(51) Int. Cl. C12N 1/14

(52) U.S. Cl. CPC C12N 1/14

(57) ABSTRACT

A method of collecting fungal spores includes steps of producing fungal spores by fungal cells within a fungal culture; combining a hydrophobic liquid with the fungal culture; mixing the fungal culture after the step of combining the hydrophobic liquid with the fungal culture, to thereby free at least a portion of the fungal spores as freed fungal spores which are freed from the fungal cells, where the freed fungal spores are partitioned within at least a portion of the hydrophobic liquid; and collecting the at least a portion of the hydrophobic liquid including the freed fungal spores.

### CROSS REFERENCE TO RELATED APPLICATIONS

This application claims the benefit of U.S. provisional patent application Ser. No. 63/399,445, filed Aug. 19, 2022, which is incorporated by reference herein.

### FIELD OF THE INVENTION

The present disclosure is directed toward a method of collecting spores by contacting the spores with a hydrophobic liquid, such as oil. The present disclosure is further directed toward cementitious materials including the collected spores.

### BACKGROUND

Spores, such as those produced by fungi, are conventionally collected from the respective microorganisms via techniques which include combining the microorganisms and spores with water. The contact of spores with water is a factor which can, at least partially, trigger premature spore germination and/or activate some enzymes in the spores.

Since the use of water alone can be ineffective, other conventional efforts include adding a surfactant to the water. Surfactants, because of their surface activities, can weaken the coat and/or plasma membrane of the spores. These effects can lower the long-term storage stability and germination ability of the spores. Even techniques which rely on surfactants tend to collect relatively lower percentages of the total spores generated in a production system.

There remains a need in the art for an improved method of spore collection.

### SUMMARY

In one aspect, a method of collecting fungal spores comprises steps of producing fungal spores by fungal cells within a fungal culture; combining a hydrophobic liquid with the fungal culture; mixing the fungal culture after the step of combining the hydrophobic liquid with the fungal culture, to thereby free at least a portion of the fungal spores as freed fungal spores which are freed from the fungal cells, where the freed fungal spores are partitioned within at least a portion of the hydrophobic liquid; collecting the at least a portion of the hydrophobic liquid including the freed fungal spores.

### DETAILED DESCRIPTION

At least certain conventional efforts to collect fungal spores from spore-producing fungal cells/mycelia include the use of water, which may include adding a surfactant to the water. Aspects of the present disclosure are directed toward improved methods of collecting fungal spores which include combining or contacting the spores with a hydrophobic liquid.

The surface of fungal spores is hydrophobic in nature. With this hydrophobic property, when combined with water, the fungal spores are hard to separate from their corresponding mycelia, thus making it difficult to collect the spores from the water. As a result, conventional attempts to collect spores with water require the use of relatively large volumes of water and nonetheless still result in low concentrations of collected spores. Moreover, the contact of spores with water and, if present, surfactants during collection can trigger one or more of unintended spore germination, unintended initial activation, and negative affect on the spore properties.

One or more aspects of the present disclosure include collecting fungal spores by utilizing a hydrophobic liquid. That is, a hydrophobic liquid can be combined with a material which includes the fungal spores, which may also be referred to as contacting the fungal spores with the hydrophobic liquid. As further discussed herein, suitable hydrophobic liquids include various oils and therefore the collection method disclosed herein can be referred to as collecting the fungal spores with oil or can be referred to as an oil-based collection method.

The collection of fungal spores via the hydrophobic liquid is expected to improve the stability of the fungal spores against harsher environmental conditions relative to fungal spores which are collected with water and a surfactant. Collection with the hydrophobic liquid also improves storage stability of the fungal spores relative to fungal spores which are collected with water and a surfactant. Still further, the hydrophobic liquid is expected to allow the spores to better separate from the producing mycelia/fungal cells, and any aqueous phase which may be present. The hydrophobic liquid is moreover not expected to be harmful to the fungal spores and corresponding fungal cells. These and/or other benefits resulting from collecting spores with the hydrophobic liquid are generally believed to arise from avoiding or minimizing contact of the spores with water, which contact with water is believed to weaken the spores.

Fungal spores are generally produced by fungal cells/mycelia within a culture, which may be referred to as a culture medium. In one or more aspects, the culture comprises fungi, which culture may therefore be referred to as a fungal culture. The culture will initially include substrate, which may be referred to as a main food or carbon source, for consumption by the fungal cells for growth of the fungal cells and/or sporulation of fungal spores. And where not all of the substrate is consumed, a subsequent culture might include residual substrate. The culture will further include the produced fungal spores until the produced fungal spores are collected. Exemplary substrate, which may be referred to as solid substrate, includes soy, rice, barley, wheat, corn, other grains, coconut, beans, other agricultural biomass, and mixtures. The substrate may be one or more of hulls, husks, or pods of these materials, including pieces thereof.

As mentioned above, the surface of fungal spores is hydrophobic in nature. Relative to the microorganism evolution and survival, this hydrophobic property enables the spores to leave nature-based aqueous systems where fungal cells generally will grow. The spores which leave these nature-based aqueous systems can therefore be carried by wind for spore dispersion to new habitats. Contrary to nature-based aqueous systems, which are inclined to spread and germinate, the use of the hydrophobic liquid of the present disclosure facilitates collection of the spores and moreover protects from unwanted early germination. Said another way, methods of the present disclosure help protect and delay the germination of the collected spores to a later time.

Relative to aspects of the present disclosure, this hydrophobic property enables spores to be collected easily and effectively from the producing cells/mycelia via combination with the hydrophobic liquid. One or more aspects include combining the hydrophobic liquid with cells/mycelia. This can include first separating the spore-containing cells/mycelia from an overall culture. Other aspects include a step of combining the hydrophobic liquid with an overall culture which includes the spores. The overall culture generally comprises fungal spores and optionally any residual substrate, residual fungal cells/mycelia, and/or residual liquids. One or more aspects include a method which is devoid of a step of separating spores from the cells/mycelia prior to the step of combining the hydrophobic liquid with the spores.

While reference is made herein to collecting the spores without utilizing water, in one or more aspects, the fungal culture can be grown in an aqueous medium. In these aspects, the particular one or more steps related to collecting the spores will generally not include adding further water. That is, the hydrophobic liquid (e.g., oil) could be added to the aqueous medium for the collection of spores therefrom. For cultures grown in an aqueous medium, an agitation tank can be used for receiving the aqueous medium, and the hydrophobic liquid can be added right to the agitation tank.

In other aspects, the fungal culture can be grown on moist solid substrate by solid state fermentation (SSF). Again, the particular one or more steps related to collecting the spores will generally not include adding further water. In these aspects, the hydrophobic liquid could be added to an SSF solid substrate bed (i.e., to an SSF vessel) for the collection of spores therefrom.

Other aspects could include continuous production of spores, such as with a conveyor and/or rotating kiln. In these aspects, the collection of the spores with the hydrophobic liquid can be on a continuous or semi-continuous basis.

For these or other potential suitable methods of producing the spores, the collection of the spores with the hydrophobic liquid offers a variety of advantages. That is, it is now understood that the spore collection method can affect the properties of the collected spores. Improving the properties of collected spores is desirable as end applications for the collected spores may rely on the ability of the spores to germinate into live cells after remaining stable for periods of time while resisting and/or tolerating environmental factors such as one or more of dryness, moisture, high temperature, low temperature, high pH, and salinity.

The collection methods disclosed herein, with the step of combining the hydrophobic liquid with the spores, and with no water or minimal water utilized for the collection step itself, are found to improve the stability of the collected spores against harsher environmental conditions relative to fungal spores which are collected with water and a surfactant. This provides the collected spores with suitable storage stability for product shelf-life. This moreover provides the collected spores with the ability to tolerate conditions that may occur during formulation and implementation.

As suggested above, the liquid which is combined with the spores for collection thereof should generally be hydrophobic. The hydrophobic surface of the fungal spores allows for improved collection with the hydrophobic liquid. The hydrophobic liquid further serves to prevent or minimize the contact of spores and cells with any aqueous phase, including for subsequent end applications of the spores. The hydrophobic liquid can further serve as a carrier liquid to enable loading of the spores into a porous substrate for use within a cementitious material, as described further herein. Moreover, the hydrophobic liquid can also serve to provide to be consumable by the fungal cells in order to support fungal cell growth and biomineralization, such as after the spores are germinated within a cracked cementitious material (e.g., concrete, mortar).

Many suitable substances and mixtures can be used for the hydrophobic liquid. Exemplary materials for the hydrophobic liquid include oils, free fatty acids, and molten fats. These include solutions and mixtures thereof. The skilled person will also understand that the term oil, as used here, can refer to compositions having a variety of chemical constituents. Exemplary oils include soybean oil, palm oil, rapeseed oil, canola oil, olive oil, sunflower oil, coconut oil, corn oil, cottonseed oil, peanut oil, safflower oil, mineral oil, paraffin oil, liquid hydrocarbons and their mixtures with chain lengths of from about C10 to about C25, and silicone oil. Exemplary free fatty acids include oleic acid, palmitic acid, stearic acid, linoleic acid, and linolenic acid. Exemplary free fatty acids may also be referred to as long chain fatty acids, which generally refers to chain lengths of from about C10 to about C25. Other suitable hydrophobic liquids include animal-based, plant-based, petroleum-derived, and synthetic hydrophobic liquids. Still other suitable hydrophobic liquids include other lipids and oil-soluble or oil-compatible compounds such as fatty alcohols, ethers, esters, glycolipids, and lipopeptides. Still other suitable materials include monoglycerides and diglycerides.

Other suitable materials for the hydrophobic liquid include those compounds with a functional group, where the compound with the functional group provides sufficiently low water solubility. Exemplary functional groups include alcohol, aldehyde, ester, amine, and amide. The sufficiently low water solubility can generally be less than 1 g/L, such as at ambient conditions. Still other suitable hydrophobic liquids include those compounds having a carboxylic acid functional group (—COOH).

The material for the hydrophobic liquid can be chosen depending on a desired end application. For example, one or more certain oils may be chosen based on also being consumable by the microorganism as a nutrient or for having suitable melting point. Moreover, one or more free fatty acids can form insoluble salts with alkali and alkaline, which would have advantages when the collected spores are utilized within a cementitious material, such as neutralizing the high pH locally and minimizing the damage to spores. Any formation of insoluble salts with alkali and alkaline, which may also be referred to as saponification, should also be balanced with the properties of the cementitious material. As suggested above, mixtures of one or more oil, one or more free fatty acid, and/or one or more molten fat might be utilized to achieve more than one of these particular benefits.

In one or more aspects, the hydrophobic liquid has a hydrophobicity which is similar or substantially similar to a surface hydrophobicity of the fungal spores. This similarity in hydrophobicity can generally serve to maximize the compatibility of the spores with the hydrophobic liquid. That is, this similarity in hydrophobicity generally maximizes the spreading and adherence of the hydrophobic liquid on the spore surface, and the dispersion of the spores in the hydrophobic liquid. This accordingly improves effectiveness in spore collection. This similarity in hydrophobicity can further serve to minimize the amount of hydrophobic liquid required to collect a desired fraction (e.g., 90%) of the spores relative to all spores present in a fungal culture. This similarity in hydrophobicity can further serve to minimize the time and/or shear/mechanical force utilized for the spore collection. In one or more aspects, the similarity in hydrophobicity is based on ambient or room temperature (e.g., 20° C. to 22° C.), though the similarity in hydrophobicity may also be adapted for different temperatures.

After combining the spores with the hydrophobic liquid, the spores can be collected in a subsequent collection step.

Relative to aspects of a collection step, fungal spores are generally denser than the hydrophobic liquid (e.g., oil). As a result, if a suspension including the spores and hydrophobic liquid is kept still for a sufficient period, generally all the spores will eventually settle down at the bottom due to different densities and gravity. This would leave a hydrophobic liquid layer above a lower layer which includes the spores. The lower layer including the settled spores can then be collected from the bottom. The time taken for particular spores to settle can differ and therefore other steps might be utilized. Fungal spores are also generally denser than water, as well as a combination of hydrophobic liquid and water, and this settling can also be utilized for suspensions including water in addition to spores and hydrophobic liquid.

In one or more aspects of the present disclosure, the spores might be collected with the hydrophobic liquid (e.g., oil) phase prior to the spores fully settling to the bottom of the hydrophobic liquid phase due to the density difference. This collection prior to complete settling might allow for relatively quicker collection of the spores. Moreover, a collected product including oil and spores may have advantageous end applications.

In one or more aspects of the present disclosure, a medium including the spores and the hydrophobic liquid can be mixed prior to a specific step of allowing the spores to settle. Examples of mixing techniques include stirring, vortexing, or paddle mixing. Mixing time and strength should be sufficient for freeing a desired amount of spores for collection thereof, while also generally avoiding damaging the spores.

The particular details of mixing a medium including the spores and the hydrophobic liquid, and subsequently collecting the spores, may depend on how the spores are produced.

As mentioned above, in one or more aspects, the fungal culture can be grown in an aqueous medium. For these aspects, after adding the hydrophobic liquid to the aqueous medium, mixing can be used to create shear to free the spores from cells/mycelia thereby resulting in freed fungal spores. The freed fungal spores will generally partition into the phase of the hydrophobic liquid, which may be referred to as the oil phase. After the mixing is stopped, the mixture generally separates into two liquid phases: an upper phase of lighter oil containing spores and a lower phase of water containing other hydrophilic biomass and substrate. The upper phase of the spore suspension in the hydrophobic liquid can then be collected.

As mentioned above, other aspects include the fungal culture being grown on moist solid substrate by solid state fermentation (SSF). For these aspects, after adding the hydrophobic liquid to the SSF solid substrate, mixing can be used to create shear to free the spores from cells/mycelia thereby resulting in freed fungal spores. The freed fungal spores will generally partition into the oil phase. After the mixing is stopped, the larger and non-hydrophobic solids (i.e., remaining substrate and biomass) can be removed. This removal can be either by filtration (e.g., screening mesh) or by allowing these materials to settle to the bottom. Where these materials are allowed to settle to the bottom, a first collection can occur for these larger materials without collecting many freed spores. The spores will be smaller than these larger materials and will therefore settle much slower than the larger pieces of remaining substrate and biomass. A second collection can then occur for the oil phase which contains the freed fungal spores. Some smaller particles of the cell and substrate debris might remain in the collected oil phase, which can be tolerable for certain end applications. In other aspects, these smaller particles of the cell and substrate debris might be further separated from the freed fungal spores.

Following the collection of a product including spores (e.g., freed fungal spores) within a hydrophobic liquid, the collected product can be further concentrated relative to the spore concentration. This can include allowing the spores of the collected product to further settle to the bottom, which may be referred to as leaving the collected product to stand. The settling step can occur in a non-mixed condition, which may also be referred to as the step of allowing the spores of the collected product to settle occurring after the mixing step. Then, after the step of allowing the spores to settle, the top layer of hydrophobic liquid from which the spores had settled, the upper liquid, which may be referred to as cleared oil, can be collected or removed. This thereby produces a remaining lower liquid which is further concentrated with the freed fungal spores relative to the original collected product. The remaining lower liquid which is further concentrated with the freed fungal spores can then be collected. Whether to utilize the settling, and how much settling to use, can depend on a desired concentration for a spore suspension relative to an intended application for the product.

Relative to a comparison with collection techniques which utilize water, the settling of the spores following collection with a hydrophobic liquid will occur relatively quicker, which may also be referred to as rapid settling of the spores. Spores will settle faster in oil than in water, for two primary reasons: density difference and hydrophobicity.

For water which contains spores, many spores would float on the top surface (i.e., air-water interface) because of the hydrophobicity. Moreover, the spores in the water would settle slowly because of a smaller density difference, as the density of the spores is not much larger than the density of water of about 1 g/cm3. In contrast, for oil which contains spores, spores will generally not remain floating on the top surface and the density difference between the spores and the oil will be larger, as oil density is about 0.9 g/cm3.

Regarding the species for the fungal cells and fungal spores to be used in aspects of the present disclosure, species for the fungal cells and fungal spores can be screened and chosen relative to the features of the present disclosure. That is, species for the fungal cells and fungal spores can be screened and chosen relative to enhancing collection thereof by combination with a hydrophobic liquid. Suitability of certain fungal cells and fungal spores may also be chosen based on an end application, such as where the collected spores are to be utilized within a cementitious material for self-repair thereof.

In aspects of the disclosure, suitable species for the fungal spores include alkalophilic and/or alkalotolerant fungi. In aspects of the disclosure, suitable species for the microorganisms and fungal spores include Scopulariopsis brevicaulis, Purpureocillium lilacinum, Myrothecium verrucaria, Aspergillus nidulans, and combinations thereof. With reference to the USDA-ARS Culture Collection (NRRL), examples include Aspergillus nidulans NRRL 187, Scopulariopsis brevicaulis NRRL 1100, Myrothecium verrucaria NRRL 2003, and Purpureocillium lilacinum NRRL 895.

The concentration of the spores in the collected product, which may be referred to as a concentrated oil suspension, will be relatively high. The concentration of spores in a collected product, prior to any specific step of settling, may be from about 1.5×10¹⁰ spores/mL to 10×10¹¹ spores/mL, or about 5×10¹⁰ spores/mL to 5×10¹¹ spores/mL, or about 1.5×10¹⁰ spores/mL to 8×10¹⁰ spores/mL, or about 1×10¹¹ spores/mL to 6×10¹¹ spores/mL, or about 2×10¹¹ spores/mL to 5×10¹¹ spores/mL.

The concentration of spores in a further concentrated product, that is, after a specific step of settling, may be about 2 times, or about 3 times, or about 5 times, or about 10 times, or about 20 times, the concentration prior to the step of settling. In one or more aspects, the concentration of spores in a further concentrated product, that is, after a specific step of settling, may be at least 2 times, or at least 3 times, or at least 5 times, or at least 10 times, or at least 15 times, the concentration prior to the step of settling.

The combination of the hydrophobic liquid with the spores can occur a relatively short time after the spores are produced by the fungal cells. This time for combining the hydrophobic liquid with the spores may also be characterized relative to when the fungal cells are added to a vessel for production of the spores. In one or more aspects, combining the hydrophobic liquid with the fungal spores occurs less than about 5 hours, or less than about 2 hours, or less than about 1 hour, or less than about 30 minutes, after the fungal spores are produced. In one or more aspects, combining the hydrophobic liquid with the fungal spores occurs less than about 5 hours, or less than about 2 hours, or less than about 1 hour, or less than about 30 minutes, or from about 5 minutes to about 30 minutes, after the fungal cells are added to a production vessel.

In one or more aspects, a mixing step occurs for less than about 2 hours, or less than about 1 hour, or less than about 30 minutes. In one or more aspects, the mixing occurs for from about 5 minutes to about 30 minutes, or from about 5 minutes to about 60 minutes, or from about 30 minutes to about 60 minutes. Mixing can utilize constant agitation/shear.

In one or more aspects, a settling step occurs for less than about 30 minutes, or less than about 15 minutes, or less than about 5 minutes. In one or more aspects, the settling occurs for from about 5 minutes to about 30 minutes, or from about 5 minutes to about 15 minutes.

In one or more aspects, the step of collecting the fungal spores includes collecting essentially all of the fungal spores which are produced. In one or more aspects, collecting the fungal spores includes collecting at least 80%, or at least 85%, or at least 90%, or at least 95%, of the fungal spores which are produced. In one or more aspects, collecting the fungal spores includes collecting about 80%, or about 85%, or about 90%, or about 95%, of the fungal spores which are produced. As mentioned above, a certain target collection amount may be utilized relative to other factors, such as the amount of oil which is utilized.

As mentioned above, one suitable end application for the collected spores is within a cementitious material. A composition for a cementitious material generally comprises a binder (e.g., cement), aggregate, water, and optionally other desirable additives as generally known to the skilled person. Cementitious materials, such as concrete, cementitious coatings, and mortar, tend to suffer cracks, which cracks can lead to a variety of undesirable effects. By incorporating the collected spores into a cementitious material, the spores will provide the ability for self-repair of the cementitious material. The cementitious materials comprising the collected spores will be capable of countering a crack through biomineralization, specifically by initiating germination of the spores, then achieving vegetative growth of fungal cells resulting from the germination, then filling in the crack with solid materials resulting from the vegetative growth.

As mentioned above, in aspects of the disclosure, the collected spores can be maintained with a portion of the hydrophobic liquid. This portion of the hydrophobic liquid can generally serve as a protective coating to protect the fungal spores from harsh conditions, which may be referred to as chemical protection.

The collected spores, which can be the coated spores, can then be impregnated or incorporated within a carrier material. The carrier material should be porous in order to impregnate or incorporate the spores therein. This inclusion of the collected spores within the carrier serves as a form of physical protection of the spores. The carrier material can receive the spores within pores therein and/or within pores on the surface. In one or more aspects, the porous carrier material is a plurality of porous particles. That is, a plurality of porous particles can be dispersed within the cementitious material. Each of the plurality of porous particles would generally include spores for repairing cracks at a variety of locations of the plurality of porous particles.

Exemplary porous carrier materials include foams. Suitable foams will generally include open cells. An exemplary foam is polyurethane. Other exemplary materials for foams include polyesters and polyamides. Other exemplary materials for foams include EPDM (ethylene propylene diene terpolymer), PVC/nitrile (polyvinyl chloride and nitrile rubber blend), ceramic, and metal (such as aluminum and nickel). Other suitable foams which have sufficient absorption and strength may also be generally known to the skilled person.

Where a porous carrier material is foam, the foam can include cement incorporated into and/or on the porous carrier material. This may include infusing, which may also be referred to as impregnating or incorporating, the cementitious component into the porous carrier material in the form of a wet cement slurry.

In addition to foams, other exemplary materials for the porous carrier material include expanded clay, celite, perlite, and expanded glass.

The porous carrier material comprising the spores can also be protected from harsh conditions via a protective coating thereon. The protective coating can include one or more free fatty acids. The protective coating can include other suitable substances such as one or more oils.

As mentioned above, the collection method disclosed herein can provide collected spores having improved properties relative to fungal spores which are collected with water and a surfactant. This may allow for longer storage times for the collected spores, where desired, relative to spores which are collected with water and a surfactant. In one or more aspects, a step of utilizing collected spores as an end product occurs from about 1 month to 2 years, or from about 2 months to 1 year, or from about 4 months to 10 months, after a step of combining the hydrophobic liquid with the spores. In other aspects, the collected spores may be utilized sooner, such as less than 1 month, or less than 2 weeks, or less than 1 week, after a step of combining the hydrophobic liquid with the spores.

In addition to the aspects related to utilizing the collected spores within a cementitious material such as concrete, other applications include foliar applications of biopesticidal spores. Collected oil suspensions including spores are advantageously expected to adhere to hydrophobic plant surfaces (e.g., leaves) and hydrophobic insect/pest surfaces (e.g., waxy outer layer) for use as biopesticide.

As suggested above, one or more aspects of the present disclosure include a method which is devoid of, or substantially devoid of, adding an aqueous solution (e.g., sterile distilled/refined water) for collecting fungal spores. As suggested above, one or more aspects include a method which is devoid of, or substantially devoid of, adding a surfactant (e.g., Tween 20, Tween 80, and Triton X-100) for collecting fungal spores.

As suggested herein, while aspects may include storing the spores which are collected with the hydrophobic liquid, it should be appreciated that the collection techniques disclosed herein are generally distinct from combining a hydrophobic liquid with already collected spores for the purpose of storing, formulating, and/or applying the spores which have already been collected. That is, one or more aspects include a method which is devoid of a specific step of collecting the spores themselves prior to combination with the hydrophobic liquid.

One or more aspects of the present disclosure include a method where the fungal spores are never in powder form. One or more aspects of the present disclosure include a method where the step of collecting occurs in a same vessel as the combination of the spores and the hydrophobic liquid. One or more aspects of the present disclosure include a method where the step of collecting occurs in a different vessel than where the combination of the spores and the hydrophobic liquid occurs.

While aspects of the disclosure are discussed above, certain exemplary Aspects are provided here.

Aspect 1. A method of collecting fungal spores, the method comprising steps of producing fungal spores by fungal cells within a fungal culture; combining a hydrophobic liquid with the fungal culture; mixing the fungal culture after the step of combining the hydrophobic liquid with the fungal culture, to thereby free at least a portion of the fungal spores as freed fungal spores which are freed from the fungal cells, where the freed fungal spores are partitioned within at least a portion of the hydrophobic liquid; and collecting the at least a portion of the hydrophobic liquid including the freed fungal spores.

Aspect 2. The method of Aspect 1, where the fungal spores and fungal cells are of a species selected from Scopulariopsis brevicaulis, Purpureocillium lilacinum, Myrothecium verrucaria, Aspergillus nidulans, and combinations thereof.

Aspect 3. The method of any of the above Aspects, wherein the fungal culture further comprises residual solid substrate which remains from the step of producing the fungal spores.

Aspect 4. The method of any of the above Aspects, wherein the method is devoid of a step of collecting the fungal spores prior to the step of combining the hydrophobic liquid with the fungal culture.

Aspect 5. The method of any of the above Aspects, where the hydrophobic liquid comprises an oil, or a free fatty acid, or a combination thereof.

Aspect 6. The method of any of the above Aspects, where the hydrophobic liquid comprises an oil selected from soybean oil, palm oil, rapeseed oil, canola oil, olive oil, sunflower oil, coconut oil, corn oil, cottonseed oil, peanut oil, safflower oil, mineral oil, paraffin oil, silicone oil, and mixtures thereof.

Aspect 7. The method of any of the above Aspects, where the hydrophobic liquid comprises a free fatty acid selected from oleic acid, palmitic acid, stearic acid, linoleic acid, linolenic acid, and mixtures thereof.

Aspect 8. The method of any of the above Aspects, where the fungal culture is within an aqueous medium.

Aspect 9. The method of any of the above Aspects, where the fungal culture is grown on moist solid substrate under solid state fermentation (SSF) conditions.

Aspect 10. The method of any of the above Aspects, further comprising steps of further concentrating the freed fungal spores by allowing the freed fungal spores to settle in a non-mixed condition, and collecting a top layer of hydrophobic liquid from which the freed fungal spores settle, thereby producing a remaining lower liquid which is further concentrated relative to the freed fungal spores.

Aspect 11. The method of Aspect 10, further comprising a step of collecting the remaining lower liquid which is further concentrated relative to the freed fungal spores.

Aspect 12. The method of any of the above Aspects, where the step of combining the hydrophobic liquid with the fungal culture is substantially devoid of adding an aqueous medium, adding a surfactant, and adding an emulsifier.

Aspect 13. The method of any of the above Aspects, where the hydrophobic liquid has a hydrophobicity substantially similar to a surface hydrophobicity of the fungal spores at room temperature of from 20° C. to 22° C.

Aspect 14. The method of any of the above Aspects, where the freed fungal spores have improved stability relative to fungal spores which are collected with water and a surfactant.

Aspect 15. The method of any of the above Aspects, where the step of collecting includes collecting at least 95% of the fungal spores produced in the step of producing.

Aspect 16. The method of any of the above Aspects, where the step of mixing comprises stirring or vortexing.

Aspect 17. The method of any of the above Aspects, further comprising a step of combining the freed fungal spores from the step of collecting with a cementitious material.

Aspect 18. The method of any of the above Aspects, further comprising a step of combining the at least a portion of the hydrophobic liquid from the step of collecting with a cementitious material.

Aspect 19. The method of any of Aspects 1 to 16, further comprising a step of applying the at least a portion of the hydrophobic liquid including the freed fungal spores from the step of collecting to a plant as a biopesticide.

In light of the foregoing, it should be appreciated that the present invention advances the art by providing an improved method of collecting spores. While particular aspects of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

Control 1—Collection with Surfactant

Fungal cells of Scopulariopsis brevicaulis were inoculated at the center of a Petri dish with potato dextrose agar. The plate was left for fungal growth and sporulation for 14 days. Spores were then aseptically collected by adding 20 mL of 1 g/L aqueous Tween 80 solution to the Petri dish and gently swirling for mixing. The spore suspension generated was transferred to a collection vial. Using this procedure, the Scopulariopsis brevicaulis spore concentration in the spore suspension collected was 2.6+/−0.5×10 6 spores/mL. This low spore concentration showed the low affinity of hydrophobic fungal spores to go into aqueous solutions even with the addition of 1 g/L Tween 80 surfactant.

Control 2—Collection with Surfactant and Scraping

The Control 1 method was repeated except that a step of scraping the culture surface with a sterile wire loop was included after adding the 20 mL aqueous Tween 80 solution to the sporulated plate. With this additional physical scraping step, the spore concentration in the collected spore suspension for S. brevicaulis increased to 10.7+/−0.3×10 9 spores/mL.

The scraping disclosed in Control 2 and elsewhere within the Examples section is utilized particularly relative to the use of cultures grown on a continuous surface of solid or semisolid material (e.g., an agar plate). In these instances, the scraping generally serves to free spores from the fungal culture. The freed spores can then be collected from the remaining culture still attached on the solid/semisolid surface. As mentioned, certain of the specific Examples include the scraping in conjunction with utilizing another component for collecting the spores.

### Example 1—Collection with Surfactant and Scraping

The Control 2 method was repeated except that 10 mL of sterile soybean oil was utilized instead of the 20 mL of the aqueous Tween 80 surfactant solution. That is, the 10 mL of soybean oil was added for collecting the S. brevicaulis spores from the sporulated culture in the Petri dish. A physical scraping step was utilized. This collection method of S. brevicaulis spores gave a concentration of 1.7+/−0.3×10¹⁰ spores/mL.

### Example 2—Settling

In an effort to further concentrate the collected S. brevicaulis spores from Example 1, Example 2 included allowing the collected spore-containing oil to settle. That is, after the material from Example 1 was collected, this collected material was left to stand for about 5 minutes for the spores to settle. This settling was observed as occurring relatively rapidly compared to other observations of spores settling from water. After the settling, the top layer of oil from which the spores had settled, which could be referred to as cleared oil, was removed. The remaining oil which contained the spores was analyzed for spore concentration. This collection and settling method for S. brevicaulis spores gave a concentration of 3.4+/−0.6×10¹¹ spores/mL.

The resulting concentrations from Control 1, Control 2, Example 1, and Example 2 are summarized in the below Table 1.

TABLE 1

| | Concentration | |
|---|---|---|
| Control 1 | 2.6 +/− 0.5 × 10⁶ | spores/mL |
| Control 2 | 10.7 +/− 0.3 × 10⁹ | spores/mL |
| Example 1 | 1.7 +/− 0.3 × 10¹⁰ | spores/mL |
| Example 2 | 3.4 +/− 0.6 × 10¹¹ | spores/mL |

Control 3—Dry Collection Via Scraping, without Utilizing Aqueous or Oil

The temperature tolerance of S. brevicaulis spores was evaluated for spores harvested dry from a potato dextrose agar plate with freshly grown and sporulated fungal culture. Sterilized Pasteur pipettes were used to gently scrape the fungal culture so that some spores became attached to the surface of pipette tips. These pipettes were placed in sterilized flasks individually, and then treated for 2 hours in a dry oven adjusted to different temperatures: 51° C., 53° C., 55° C., 60° C., 65° C., and 70° C. For each temperature, 4 Pasteur pipettes were used. Fresh potato dextrose agar plates were poked with these pipettes to inoculate the heat-treated spores. The inoculation was done inside a laminar flow hood under a flow of filter-sterilized air. The inoculated plates were kept at room temperature for observation of spore germination at these inoculated spots. Results showed that the spores treated at all tested temperatures, up to 70° C., could germinate. The S. brevicaulis spores collected dry were found to tolerate at least 70° C. for 2 hours under the dry heat condition used in our test procedure.

While these dry collected spores were shown to have this tolerance at all tested temperatures, dry collection of spores is generally not practical for large-scale collection. For example, a dry collection procedure only moves the spores to the surface of the dry object used to scrape the culture, and the spores would need to be collected from the surface. And only a very small fraction of produced spores can be collected by this technique, without using a deleteriously large number of the scraping objects to scrape the culture repeatedly. Further, for certain end applications, the storage or utilization of collected spores which are attached on the surface of scraping objects is highly impractical or implausible.

Control 4—Collection with Surfactant and Scraping

The Control 3 method was generally repeated except for using an aqueous surfactant solution (1 g/L Tween 80) for the collection, and the collected S. brevicaulis spores were treated at 60° C., 65° C., and 70° C. in a dry oven for 2 hours. The germination ability of the spores collected using the aqueous surfactant solution was poor. The spores treated for 2 hours at 60° C. struggled to germinate; 3 out of 5 inoculation points germinated. Those treated at 65° C. and 70° C. could not germinate at all.

### Example 5—Collection with Soybean Oil and Scraping

The Control 3 method was generally repeated except for using soybean oil for the collection, and the collected S. brevicaulis spores were treated at 60° C., 65° C., and 70° C. in a dry oven for 2 hours. For the oil-collected spores, those treated at 60° C. germinated rapidly, which was comparable to the dry collected spores in Control 3 at this temperature. Those treated at 65° C. and 70° C. also germinated, but the germination occurred after 7 days, which was later than the 3 days observed for dry collected spores in Control 3. However, this germination was improved relative to Control 4 with collection using an aqueous surfactant solution. Again, as mentioned above, dry collection of spores is generally not practical for large scale collection.

### Example 6—Stability of Spores within Mixture of Free Fatty Acids and Soybean Oil and within Soybean Oil

Stability of S. brevicaulis spores was tested after the spores had been in contact with a mixture of free fatty acids and soybean oil, containing 9 g oleic acid, 3 g stearic acid, 2 g palmitic acid, and 3 mL soybean oil, for 2 months. Spores kept in soybean oil for 51 and 93 days were also tested. These spores were inoculated onto potato dextrose agar plates. All inoculations germinated, demonstrating the long-term spore stability of spores in an oil suspension.

### Example 7—Stability of Spores in Porous Carrier Particles

The S. brevicaulis spores in soybean oil and in the oil and free fatty acids mixture from Example 6 were loaded into porous carrier particles and then placed in a wet mortar mixture. One more system had the porous carrier particles loaded with spores in soybean oil, and then was placed in a wet mortar mixture. The porous carrier particles were made of polyurethane foam and included cement incorporated therein for strength. The loaded polyurethane foam particles were coated with a thin layer of the oil and free fatty acids mixture on the surface of the carrier particles before being placed in wet mortar. The wet mortar had a 0.5:1:2.75 weight ratio of water:cement:sand to create the mortar. During the curing period, the mortar is believed to have had a pH of >12.

After the mortar with the particles was cured and stored for different amounts of days, the samples were broken, and the spores-laden carrier particles were placed on potato dextrose agar plates to examine spore germination. Even after being subjected to the harsh, wet mortar environment, the spores in the soybean oil were found to germinate after 8, 51, 66, 106, 335, and 569 days (longer time not tested); the spores in the oil and free fatty acids mixture germinated after 17, 36, 51, 66, 81, 93, 106, 116, 133, 153, 213, 335, and 569 days (longer time not tested); and the spores in oil with oil and free fatty acids coated carrier particles germinated after at least 7 months (longer time not tested).

Various modifications and alterations that do not depart from the scope and spirit of this invention will become apparent to those skilled in the art. This invention is not to be duly limited to the illustrative aspects set forth herein.

What is claimed is:

1. A method of collecting fungal spores, the method comprising steps of
producing fungal spores by fungal cells within a fungal culture;
combining a hydrophobic liquid with the fungal culture;
mixing the fungal culture after the step of combining the hydrophobic liquid with the fungal culture, to thereby free at least a portion of the fungal spores as freed fungal spores which are freed from the fungal cells, where the freed fungal spores are partitioned within at least a portion of the hydrophobic liquid; and
collecting the at least a portion of the hydrophobic liquid including the freed fungal spores.

2. The method of claim 1, where the fungal spores and fungal cells are of a species selected from Scopulariopsis brevicaulis, Purpureocillium lilacinum, Myrothecium verrucaria, Aspergillus nidulans, and combinations thereof.

3. The method of claim 1, wherein the fungal culture further comprises residual solid substrate which remains from the step of producing the fungal spores.

4. The method of claim 1, wherein the method is devoid of a step of collecting the fungal spores prior to the step of combining the hydrophobic liquid with the fungal culture.

5. The method of claim 1, where the hydrophobic liquid comprises an oil, or a free fatty acid, or a combination thereof.

6. The method of claim 1, where the hydrophobic liquid comprises an oil selected from soybean oil, palm oil, rapeseed oil, canola oil, olive oil, sunflower oil, coconut oil, corn oil, cottonseed oil, peanut oil, safflower oil, mineral oil, paraffin oil, silicone oil, and mixtures thereof.

7. The method of claim 1, where the hydrophobic liquid comprises a free fatty acid selected from oleic acid, palmitic acid, stearic acid, linoleic acid, linolenic acid, and mixtures thereof.

8. The method of claim 1, where the fungal culture is within an aqueous medium.

9. The method of claim 1, where the fungal culture is grown on moist solid substrate under solid state fermentation (SSF) conditions.

10. The method of claim 1, further comprising steps of further concentrating the freed fungal spores by allowing the freed fungal spores to settle in a non-mixed condition, and collecting a top layer of hydrophobic liquid from which the freed fungal spores settle, thereby producing a remaining lower liquid which is further concentrated relative to the freed fungal spores.

11. The method of claim 10, further comprising a step of collecting the remaining lower liquid which is further concentrated relative to the freed fungal spores.

12. The method of claim 1, where the step of combining the hydrophobic liquid with the fungal culture is substantially devoid of adding an aqueous medium, adding a surfactant, and adding an emulsifier.

13. The method of claim 1, where the hydrophobic liquid has a hydrophobicity substantially similar to a surface hydrophobicity of the fungal spores at room temperature of from 20° C. to 22° C.

14. The method of claim 1, where the freed fungal spores have improved stability relative to fungal spores which are collected with water and a surfactant.

15. The method of claim 1, where the step of collecting includes collecting at least 95% of the fungal spores produced in the step of producing.

16. The method of claim 1, where the step of mixing comprises stirring or vortexing.

17. The method of claim 1, further comprising a step of combining the freed fungal spores from the step of collecting with a cementitious material.

18. The method of claim 1, further comprising a step of combining the at least a portion of the hydrophobic liquid from the step of collecting with a cementitious material.

19. The method of claim 1, further comprising a step of applying the at least a portion of the hydrophobic liquid including the freed fungal spores from the step of collecting to a plant as a biopesticide.
